(19) United States
(12) Patent Application Publication
(10) Pub. No.: US 2003/0105170 A1
(43) Pub. Date: Jun. 5, 2003

(54) Surface area of cobalt catalyst supported by silica carrier material

(75) Inventors: Kandaswamy Jothimurugesan, Ponca City, OK (US); Rafael L. Espinoza, Ponca City, OK (US); Nithya Srinivasan, Ponca City, OK (US)

Correspondence Address: DAVID W. WESTPHAL, CONOCO PHILLIPS, P.O. BOX 1267, PONCA CITY, OK, 74602-1267, US

(73) Assignee: Conoco Inc., Houston, TX

(21) Appl. No.: 10/252,620

(22) Filed: Sep. 23, 2002

Related U.S. Application Data
(60) Provisional application No. 60/323,916, filed on Sep. 21, 2001.

(51) Int. Cl. C07C 27/06; B01J 21/08

(52) U.S. Cl. 518/715; 502/260; 502/241; 502/243

(57) ABSTRACT

The present invention teaches a method for increasing the cobalt surface area per gram of catalyst in a cobalt Fischer-Tropsch catalyst, supported on a silica-based carrier material, by using cobalt amine carbonate precursors. A Fischer-Tropsch catalyst preferably includes a catalytically active first metal containing cobalt, and a carrier material containing silica or a silica compound with a cobalt surface area greater than 13 m²/g catalyst. The catalyst active in the FT reaction has a minimum alpha value of 0.87 and a CO conversion of 24 wt % or more. In accordance with another preferred embodiment, a process for producing a Fischer-Tropsch catalyst includes saturating silica or silica compounds with a solution of cobalt amine carbonate, removing the excess solution by filtration, heating the resulting product in order to allow cobalt hydroxycarbonate to precipitate, and drying and calcining the resulting product. Optionally the calcined product is reduced.

### CROSS-REFERENCE TO RELATED APPLICATIONS

The present application claims benefit of U.S. Application Serial No. 60/323,916, filed Sep. 21, 2001, and entitled “Improved Surface Area of Cobalt Catalyst Supported By Silica Carrier Material,” which is incorporated herein by reference.

### STATEMENT REGARDING FEDERALLY SPONSORED RESEARCH OR DEVELOPMENT

Not applicable.

### TECHNICAL FIELD OF THE INVENTION

The present invention relates to a process for the preparation of hydrocarbons from synthesis gas, i.e., a mixture of carbon monoxide and hydrogen, typically labeled the Fischer-Tropsch process. More particularly, this invention relates to Fischer-Tropsch catalysts including cobalt. Still more particularly, the present invention relates to reducing the cobalt content in Fischer-Tropsch catalysts by using cobalt amine carbonate precursors while increasing the cobalt surface area.

### BACKGROUND OF THE INVENTION

Large quantities of methane, the main component of natural gas, are available in many areas of the world, and natural gas is predicted to outlast oil reserves by a significant margin. However, most natural gas is situated in areas that are geographically remote from population and industrial centers. The costs of compression, transportation, and storage make its use economically unattractive. To improve the economics of natural gas use, much research has focused on the use of methane as a starting material for the production of higher hydrocarbons and hydrocarbon liquids, which are more easily transported and thus more economical. The conversion of methane to hydrocarbons is typically carried out in two steps. In the first step, methane is converted into a mixture of carbon monoxide and hydrogen (i.e., synthesis gas or syngas). In a second step, the syngas is converted into hydrocarbons.

This second step, the preparation of hydrocarbons from synthesis gas, is well known in the art and is usually referred to as Fischer-Tropsch synthesis, the Fischer-Tropsch process, or Fischer-Tropsch reaction(s). Fischer-Tropsch synthesis generally entails contacting a stream of synthesis gas with a catalyst under temperature and pressure conditions that allow the synthesis gas to react and form hydrocarbons. More specifically, the Fischer-Tropsch reaction is the catalytic hydrogenation of carbon monoxide to produce any of a variety of products ranging from methane to higher alkanes and aliphatic alcohols. Research continues on the development of more efficient Fischer-Tropsch catalyst systems and reaction systems that increase the selectivity for high-value hydrocarbons in the Fischer-Tropsch product stream.

There are continuing efforts to find catalysts that are more effective at producing these desired products. Product distribution, product selectivity, and reactor productivity depend heavily on the type and structure of the catalyst and on the reactor type and operating conditions. It is particularly desirable to maximize the production of high-value liquid hydrocarbons, such as hydrocarbons with five or more carbon atoms per hydrocarbon chain (C₅₊).

Catalyst supports for catalysts used in Fischer-Tropsch synthesis of hydrocarbons have typically been oxides. Alumina is widely used as a metal catalyst support because it has a high surface area and porosity, which allows for high dispersion of a catalytic metal. However, at high temperatures, vacancies occurring in sub-surface layers induce motion of the surface ions because the oxygen ions that might be bonded to the vacant ions are instead free to move. Such a movement can initiate sintering and phase transformation that lead to a decrease in surface area of the alumina. Further, movement of the surface ions in a support may lead to catalyst diffusion into the support. Cobalt, for example, is known to migrate into the lattice sites of alumina and form aluminates. Aluminates are undesirable because they are known to be resistant to reduction and lower the catalyst activity.

The use of iron and/or cobalt together with one or more promoters and a support, for Fischer-Tropsch catalysis is well known. For natural gas derived syngas, certain advanced cobalt catalysts have been proven to be very effective for Fischer-Tropsch synthesis. However, for these catalysts, extensive promotion with noble and/or near noble metals has been required; cobalt is typically present in concentrations of about 20 wt %. These high concentrations are necessary because the best incipient wetness technique on a commercial support typically yields surface area in the range of 8 to 12 m²/g of catalyst. Due in significant part to the cost of obtaining and adding such promoters, and high concentrations of cobalt, these advanced cobalt catalysts have typically been quite expensive. U.S. Pat. No. 5,874,381 describes a technique that mixes cobalt amine carbonate precursors in a slurry of transition alumina to form a high cobalt surface area catalyst on an alumina support. However, as described above, alumina inherently has many drawbacks for use as a support. Thus, a need presently exists for an inexpensive means of preparing a high cobalt surface area Fischer-Tropsch catalyst on a support other than alumina.

### SUMMARY OF THE INVENTION

The present invention provides silica-based supported cobalt catalysts with very high cobalt surface areas per gram of catalysts. Very high metal (or cobalt) surface area per gram catalyst is defined herein as at least 13 m²/g. In Fischer-Tropsch reactions, as generally in hydrogenation reactions, the active phase of cobalt is the metallic phase. In these catalysts the useful cobalt atoms are those that are exposed at the surface of the cobalt particles. The cobalt atoms that are not exposed (i.e. not at the surface) will not participate in catalytic reaction. Because cobalt is an expensive metal, it is particularly desirable to maximize the ratio relating the number of exposed cobalt atoms to the total number of cobalt atoms in the catalyst. This corresponds to an increase in the cobalt surface area per gram of cobalt.

A silica-containing compound is preferred as the carrier material for a number of reasons. The degree of reduction of cobalt is generally higher on silica than on alumina, allowing for cobalt in silica supports to be potentially more active. Silica is also considered to be more inert than alumina. This is desirable, because as described in detail above, catalyst interactions with a carrier material can lead to catalyst migration into the carrier material, ultimately decreasing the number of cobalt atoms participating in the desired catalytic reaction. Further, silica has a low methane selectivity. As is well known, methane selectivity should be minimized in order to ensure that the production of high-value liquid hydrocarbons, such as C₅₊, is maximized.

In accordance with a preferred embodiment, a process for producing hydrocarbons includes contacting a feed stream of hydrogen and carbon monoxide with a catalyst in a reaction zone maintained at conversion-promoting conditions effective to produce an effluent stream of hydrocarbons, where the catalyst includes a catalytically active first metal containing cobalt, and a carrier material containing silica or a silica compound.

In accordance with another preferred embodiment, a Fischer-Tropsch catalyst includes a catalytically active first metal containing cobalt, and a carrier material containing silica or a silica compound.

In accordance with yet another preferred embodiment, a process for producing a Fischer-Tropsch catalyst includes saturating silica or silica compounds with a solution of cobalt amine carbonate, removing the excess solution by filtration, heating the resulting product in order to allow cobalt hydroxycarbonate to precipitate, and drying and calcining the resulting product.

The catalyst according to any of the above embodiments of the present invention may optionally include a second metal selected from the group of promoters including Ru, Re, Pt, Ag, B, and any combinations thereof. Additionally, the catalyst may have a cobalt surface area of at least 16 m² per gram catalyst.

### DETAILED DESCRIPTION OF THE PREFERRED EMBODIMENTS

The present cobalt Fischer-Tropsch catalysts are preferably prepared by impregnation and/or precipitation techniques using cobalt amine carbonate precursors to increase cobalt content dispersion, while decreasing overall cobalt content. The amine carbonate precursors increase the cobalt surface area per gram of cobalt, so activity levels are maintained while the cost is considerably decreased. According to the present invention, the cobalt surface area per gram of catalyst is preferably maintained at greater than 13 m²/g.

Many catalyst types are produced by impregnation. Impregnation includes the repeated dipping of a porous support into a solution containing a desired catalytic agent. The agent must be applied uniformly in a predetermined quantity to a preset depth of penetration. This is especially true of catalysts based on noble metals. However, the liquid penetration into the support is hindered by air trapped in the pores. As a result, various techniques like pressurizing, vacuum treatment, acoustic activation, etc. are used to facilitate the impregnation process.

Similarly, many catalysts are subjected to precipitation. Precipitation employs the formation of a separable solid substance from a solution, either by converting the substance into an insoluble form or by changing the composition of the solvent to diminish the solubility of the substance in it. The distinction between precipitation and crystallization lies largely in whether emphasis is placed on the process by which the solubility is reduced or on that by which the structure of the solid substance becomes organized.

In attempts to precipitate a single substance from a solution containing several components, undesired constituents often are incorporated in the crystals, reducing their purity and impairing the accuracy of the analysis. Such contamination can be reduced by carrying out the operations with dilute solutions and by adding the precipitating agent slowly; an effective technique is that called homogeneous precipitation, in which the precipitating agent is synthesized in the solution rather than added mechanically. In difficult cases it may be necessary to isolate an impure precipitate, redissolve it, and reprecipitate it; most of the interfering substances are removed in the original solution, and the second precipitation is performed in their absence.

According to the present invention, cobalt Fischer Tropsch catalysts may be prepared by an impregnation technique, a precipitation technique, or a combination of techniques. In preferred embodiments, at least one catalytically active metal is deposited, via impregnation, precipitation, or both, on a support. The metal can be any metal that is effective for Fischer-Tropsch synthesis, and preferably comprises approximately 5-20 wt. % cobalt. The support is preferably a porous silica-containing material. The silica-containing compound can be any suitable compound, including but not limited to silica, silica-titania, silica-alumina, silica-zirconia, silica-vanadia, and silica-magnesia.

The present invention includes a technique that mixes cobalt amine carbonate precursors in a slurry of a silica-containing compound to form a high cobalt surface area Fischer-Tropsch catalyst on a silica-based support. It is believed that using cobalt amine carbonate precursors to produce Fischer Tropsch catalysts will result in increased cobalt dispersion in the catalysts. This is because the impregnation solution is a diluted suspension, allowing cobalt atoms to move freely throughout the solution, without settling at the bottom.

In a first embodiment, an impregnation solution is formed by combining cobalt and ammonium carbonate with ammonium hydroxide and demineralized water. In a second step, the impregnation solution of step 1 is combined with a silica-containing compound the mixture is heated to a temperature of at least about 80° C. in order to allow cobalt hydroxycarbonate to precipitate, and the resulting material then is dried to form a silica-based supported cobalt catalyst.

For example, the catalyst may be prepared by the following method:

Step 1

Weigh out 900 g of ammonium hydroxide solution (28% NH₃ in water) and add 42 g of demineralized water. Add 150 g of ammonium carbonate and begin stirring. Heat gently to 35° C. to assist dissolving the powder. When fully dissolved, add slowly 141 g of basic cobalt carbonate. Continue stirring for approximately 2 hours. Filter.

Step 2

Weigh out 104 g of silica into a beaker and add 125 mL of the impregnation solution. After 10 minutes put the impregnated granules or extrudates on a filter to drain excess liquid. Dry the product for 1 hour at room temperature, then 1 hour at 80° C., and overnight (16 hours) at 120° C. Calcine the dried product in an air flow at 350° C. for 2 hours using a rotary calciner. Optionally, the calcination temperature may be increased up to 900° C. to reduce the calcination time so that the calcination time is at most 2 hours.

In a preferred embodiment, the combined silica and impregnation solution are heated to a temperature of at least 80° C. and more preferably between 80° C. and 120° C. to enhance deposition of the catalytic metal on the silica.

Alternately, in another preferred embodiment, cobalt Fischer Tropsch catalysts may be prepared using a precipitation technique, where the cobalt-amine precursors are initially part of a solution. Still further, in another preferred embodiment, cobalt Fischer Tropsch catalysts may be prepared by a combination or series of impregnation and precipitation techniques. For example, a portion of the FT active metal can be deposited by precipitation to obtain a good metal dispersion, with the rest of the active metal being deposited in a second step by other standard impregnation techniques such as incipient wetness impregnation.

The resulting catalyst should have good mechanical stability at the conversion-promoting conditions in which it is to be used. In some embodiments, the desired mechanical stability of the catalyst can be achieved through an optional pre-treatment of the carrier material comprising silica or a silica compound. The pre-treatment of the carrier material can be done using one or more of the following techniques: calcination, addition of at least one structural promoter, and chemical treatment. It should be understood that any suitable pre-treatment technique that increases mechanical strength of the catalyst can be used, and the list of techniques stated above is not intended to limit the scope of the invention. Calcination of the carrier material is preferably carried out in the presence of air or oxygen at a temperature between 200 to 900° C. The addition of at least one structural promoter is preferably done by precipitation or impregnation of a structural promoter precursor with the carrier material.

The impregnated support may be dried if desired, and is preferably reduced with hydrogen or a hydrogen containing gas. The hydrogen reduction step may not be necessary if the catalyst is prepared with zero-valent cobalt. In another preferred method, the impregnated support is dried, oxidized with air or oxygen and reduced in the presence of hydrogen.

Typically, at least a portion of the metal(s) of the catalytic metal component (a) of the catalysts of the present invention is present in a reduced state (i.e., in the metallic state). Therefore, it is normally advantageous to activate the catalyst prior to use by a reduction treatment, in the presence of hydrogen at an elevated temperature. In some embodiments, the catalyst may be treated with hydrogen at a temperature in the range of from about 75° C. to about 500° C., for about 0.5 to about 36 hours at a pressure of about 1 to about 75 atm. Pure hydrogen may be used in the reduction treatment, as may a mixture of hydrogen and an inert gas such as nitrogen, or a mixture of hydrogen and other gases as are known in the art, such as natural gas, methane, light hydrocarbons, carbon monoxide and carbon dioxide. Reduction with pure hydrogen and reduction with a mixture of hydrogen and carbon monoxide are preferred. The amount of hydrogen may range from about 1% to about 100% by volume.

Operation

A source according to a preferred embodiment of the present invention is preferably used as a catalyst in the Fischer-Tropsch process for catalytic hydrogenation of carbon monoxide. The feed gases charged to the reaction process of the invention comprise hydrogen, or a hydrogen source, and carbon monoxide. Hydrogen/carbon monoxide mixtures suitable as a feedstock for conversion to hydrocarbons according to the process of this invention can be obtained from light hydrocarbons such as methane by means of steam reforming, partial oxidation, or other processes known in the art. Preferably the hydrogen is provided by free hydrogen, although some Fischer-Tropsch catalysts have sufficient water gas shift activity to convert some water to hydrogen for use in the Fischer-Tropsch process. It is preferred that the molar ratio of hydrogen to carbon monoxide in the feed be greater than 0.5:1 (e.g., from about 0.67 to 2.5). Preferably, the feed gas stream contains hydrogen and carbon monoxide in a molar ratio from about 1.8 to about 2:3. The feed gas may also contain carbon dioxide. The feed gas stream should contain a low concentration of compounds or elements that have a deleterious effect on the catalyst, such as poisons. For example, the feed gas may need to be pre-treated to ensure that it contains low concentrations of sulfur or nitrogen compounds such as hydrogen sulfide, hydrogen cyanide, ammonia and carbonyl sulfides.

The feed gas is contacted with the catalyst in a reaction zone. Mechanical arrangements of conventional design may be employed as the reaction zone including, for example, fixed bed, fluidized bed, slurry phase, slurry bubble column, reactive distillation column, or ebullating bed reactors, among others, may be used. Accordingly, the size and physical form of the catalyst particles may vary depending on the reactor in which they are to be used.

The Fischer-Tropsch process is typically run in a continuous mode. In this mode, the gas hourly space velocity through the reaction zone typically may range from about 50 to about 10,000 hr⁻¹, preferably from about 300 hr⁻¹ to about 2,000 hr⁻¹. The gas hourly space velocity is defined as the volume of reactants per time per reaction zone volume. The volume of reactant gases is at standard conditions defined by a pressure of 1 atm (101 kPa) and a temperature of 0° C. (273.16 K). The reaction zone volume is defined as the portion of the reactor vessel volume in which the reaction takes place and which is occupied by a gaseous phase comprising reactants, products and/or inerts; a liquid phase comprising liquid/wax products and/or other liquids; and a solid phase comprising catalyst. The reaction zone temperature is typically in the range from about 160° C. to about 300° C. Preferably, the reaction zone is operated at conversion promoting conditions at temperatures from about 190° C. to about 260° C. The reaction zone pressure is typically in the range of about 80 psia (552 kPa) to about 1000 psia (6895 kPa), more preferably from 80 psia (552 kPa) to about 600 psia (4137 kPa), and still more preferably, from about 140 psia (965 kPa) to about 500 psia (3447 kPa).

The products resulting from the process will have a great range of molecular weights. Typically, the carbon number range of the product hydrocarbons will start at methane and continue to about 50 to 100 carbons per molecule or more. The process is particularly useful for making hydrocarbons having five or more carbon atoms especially when the above-referenced preferred space velocity, temperature and pressure ranges are employed.

The wide range of hydrocarbons produced in the reaction zone will typically afford liquid phase products at the reaction zone operating conditions. Therefore the effluent stream of the reaction zone will often be a mixed phase stream including liquid and vapor phase products. The effluent stream of the reaction zone may be cooled to effect the condensation of additional amounts of hydrocarbons and passed into a vapor-liquid separation zone separating the liquid and vapor phase products. The vapor phase material may be passed into a second stage of cooling for recovery of additional hydrocarbons. The liquid phase material from the initial vapor-liquid separation zone together with any liquid from a subsequent separation zone may be fed into a fractionation column. Typically, a stripping column is employed first to remove light hydrocarbons such as propane and butane. The remaining hydrocarbons may be passed into a fractionation column where they are separated by boiling point range into products such as naphtha, kerosene and fuel oils. Hydrocarbons recovered from the reaction zone and having a boiling point above that of the desired products may be passed into conventional processing equipment such as a hydrocracking zone in order to reduce their molecular weight. The gas phase recovered from the reactor zone effluent stream after hydrocarbon recovery may be partially recycled if it contains a sufficient quantity of hydrogen and/or carbon monoxide.

Without further elaboration, it is believed that one skilled in the art can, using the description herein, utilize the present invention to its fullest extent. The following examples are to be construed as illustrative, and not as constraining the scope of the present invention in any way.

### EXAMPLES

### Example 1

A cobalt Fischer-Tropsch catalyst was prepared by impregnation at about 80° C. using cobalt amine carbonate precursors. A silica support having an average pore diameter of 53 Å was used. The BET surface area of the support was 533 m²/g and the pore volume was 0.89 cc/g. The catalyst was prepared having 17.7 wt % cobalt. The catalyst was calcined at 350° C. for 2 hours. Hydrogen chemisorption was used to calculate cobalt surface area per gram of catalyst. Results are shown in Table 1.

### Example 2

A cobalt Fischer-Tropsch catalyst was prepared by impregnation at about 80° C. using cobalt amine carbonate precursors. A silica support having an average pore diameter of 123 Å was used. The BET surface area of the support was 292 m²/g and the pore volume was 1.04 cc/g. The catalyst was prepared having 16.8 wt % cobalt. The catalyst was calcined at 350° C. for 2 hours. Hydrogen chemisorption was used to calculate cobalt surface area. Results are shown in Table 1.

### Example 3

A cobalt Fischer-Tropsch catalyst was prepared by impregnation at about 80° C. using cobalt amine carbonate precursors. A silica support having an average pore diameter of 53 Å was used. The BET surface area of support was 533 m²/g and the pore volume was 1.04 cc/g. The catalyst was prepared having 17.7 wt % cobalt and 0.02 wt % Pt. The catalyst was calcined at 350° C. for 2 hours. Hydrogen chemisorption was used to calculate cobalt surface area per gram of catalyst. Results are shown in Table 1.

TABLE 1

| | | | Cobalt | |
|---|---|---|---|---|
| | | | Surface Area, | Dispersion, |
| Example | Catalyst | Co wt % | m²/g | % |
| 1 | Co/SiO₂ | 17.7 | 25.8 | 21.5 |
| 2. | Co/SiO₂ | 16.8 | 19.2 | 16.9 |
| 3. | Co/0.02% Pt/SiO₂ | 17.1 | 16.2 | 13.5 |

General Procedure for Continuous Tests

The catalyst test unit was composed of a syngas feed system, a tubular reactor, which had a set of wax and cold traps, back pressure regulators, and three gas chromatographs (one on-line and two off-line).

Carbon monoxide was purified before being fed to the reactor over a 22% lead oxide on alumina catalyst placed in a trap to remove any iron carbonyls present. The individual gases or mixtures of the gases were mixed in a 300 mL vessel filled with glass beads before entering the supply manifold feeding the reactor.

The reactor was made of ⅜ in. (0.95 cm) outer diameter by ¼ in. (0.63 cm) inner diameter stainless steel tubing. The length of the reactor tubing was 14 in. (35.6 cm). The actual length of the catalyst bed was 10 in. (25.4 cm) with 2 in. (5.1 cm) of 25/30 mesh (0.71/0.59 mm) glass beads and glass wool at the inlet and outlet of the reactor.

The wax and cold traps were made of 75 mL pressure cylinders. The wax traps were set at 140° C. while the cold traps were set at 0° C. The reactor had two wax traps in parallel followed by two cold traps in parallel. At any given time products from the reactor flowed through one wax and one cold trap in series. Following a material balance period, the hot and cold traps used were switched to the other set in parallel, if needed. The wax traps collected a heavy hydrocarbon product distribution (usually between C₆ and above) while the cold traps collected lighter hydrocarbon product distribution (usually between C₃ and C₂₀). Water, a major product of the Fischer-Tropsch process, was collected in both traps.

General Analytical Procedure

The uncondensed gaseous products from the reactors were analyzed using a common online HP Refinery Gas Analyzer. The Refinery Gas Analyzer was equipped with two thermal conductivity detectors and measured the conditions of CO, H₂, N₂, CH₄, C₂ to C₅, alkenes/alkanes/isomers, and water in the uncondensed reactor products. The products from each of the hot and cold traps were separated into an aqueous and an organic phase. The organic phase from the hot trap was usually solid at room temperature. A portion of this solid product was dissolved in carbon disulfide before analyzed. The organic phase from the cold trap was usually liquid at room temperature and was analyzed as obtained. The aqueous phase of the two traps was combined and analyzed for alcohols and other oxygenates. Two offline gas chromatographs equipped with flame ionization detectors were used for the analysis of the organic and aqueous phases collected from the wax and cold traps.

Catalyst Testing Procedure

3 grams of catalyst was mixed with 4 grams of 25/30 mesh (0.71/0.59 mm) and 4 grams of 2 mm glass beads. The 14 in. (35.6 cm) tubular reactor was loaded with 25/30 mesh (0.71/0.59 mm) glass beads so as to occupy 2 in. (5.1 cm) length of the reactor. The catalyst/glass bead mixture was then loaded and occupied 10 in. (25.4 cm) of the reactor length. The remaining 2 in. (5.1 cm) of reactor length was once again filled with 25/30 mesh (0.71/0.59) glass beads. Both ends of the reactor were plugged with glass wool.

Catalyst activation was subsequently carried out using the following procedure. The reactor was heated to 120° C. under nitrogen flow (100 cc/min) and 40 psig (377 kPa) at a rate 1.5° C./min. The reactor was maintained at 120° C. under these conditions for two hours for drying of the catalyst. At the end of the drying period, the flow was switched from nitrogen to hydrogen. The reactor was heated under hydrogen flow (100 cc/min) and 40 psig (377 kPa) at a rate 1.4° C./min to 400° C. The reactor was maintained at 400° C. under these conditions for sixteen hours for catalyst reduction. At the end of the reduction period, the flow was switched back to nitrogen and the reactor cooled to reaction temperature (220° C).

The reactor was pressurized to the desired reaction pressure and cooled to the desired reaction temperature. Syngas, with a 2:1 H₂/CO ratio was then fed to the reactor.

The first material balance period started approximately four hours after the start of the reaction. A material balance period lasted approximately 17 to 24 hours. During the material balance period, data was collected for feed syngas and exit uncondensed gas flow rates and compositions, weights and compositions of aqueous and organic phases collected in the wax and cold traps, and reaction conditions (i.e. temperature and pressure). The information collected was then analyzed for total, as well as individual carbon, hydrogen and oxygen material balances. From this information, CO conversion (%), selectivity/alpha plot for all (C₁ to C₄₀) of the hydrocarbon products, C₅⁺ productivity (g/hr/kg cat), weight percent CH₄ in hydrocarbon products (%), and other desired reactor outputs were calculated.

The results obtained from the continuous-flow Fischer-Tropsch catalysts testing unit is shown in Table 2. Table 2 lists the catalyst composition, CO conversion (%), Alpha value from the Anderson-Shultz-Flory plot of the hydrocarbon product distribution, C₅⁺ Productivity (g C₅⁺/hour/kg catalyst), and weight percent methane in the total hydrocarbon product (%). The temperature was 220° C., the pressure was approximately 340 psig (2445 kPa) to 362 psig (2597 kPa), and the space velocity of the reactant gases was 6 NL/hour/g cat.

TABLE 2

| Example | Catalyst | % Conv. | Alpha | C₅⁺ | % C₁ |
|---|---|---|---|---|---|
| 1 | 17.7% Co/SiO₂ | 27 | 0.89 | 203 | 27 |
| 2 | 16.8% Co/SiO₂ | 36 | 0.88 | 306 | 19 |
| 3. | 17.7% Co/0.02% Pt/SiO₂ | 24 | 0.87 | 201 | 20 |

What is claimed is:

1. A process for producing hydrocarbons, comprising contacting a feed stream comprising hydrogen and carbon monoxide with a catalyst in a reaction zone maintained at conversion-promoting conditions effective to produce an effluent stream comprising hydrocarbons, wherein the catalyst comprises:
a catalytically active first metal comprising cobalt; and
a carrier material comprising silica or a silica compound;
wherein the catalyst has a cobalt surface area per gram catalyst of at least 13 m²/g.

2. The process according to claim 1 wherein the catalyst has an alpha of at least 0.87.

3. The process according to claim 1 wherein the catalyst has a CO conversion of at least 24%.

4. The process according to claim 1 wherein the catalyst has a cobalt surface area per gram catalyst of at least 16 m²/g.

5. The process according to claim 1 wherein the catalyst is made by the steps of:
a) providing a cobalt precursor in a solution;
b) contacting the solution with a silica-containing support material for a period of time sufficient to allow a desired amount of cobalt to be deposited on the support material;
c) allowing the cobalt-deposited support material to dry; and
d) calcining the dried cobalt-deposited support to generate a cobalt-deposited silica-based catalyst; and
e) optionally, reducing the cobalt-deposited silica-based catalyst.

6. The process according to claim 5 wherein step c) is carried out between 25° C. and 120° C.

7. The process according to claim 5 wherein the wherein step b) lasts between 1 and 20 minutes.

8. The process according to claim 5 wherein the wherein step b) is carried out at a temperature of at least about 80° C.

9. The process according to claim 5 wherein the wherein step b) is carried out at a temperature between about 80° C. and 120° C.

10. The process of claim 5 wherein calcination occurs at a temperature of between 200° C. and 900° C.

11. The process of claim 5 wherein calcination occurs at a temperature of between 275° C. and 350° C.

12. The process of claim 5 wherein calcination preferably occurs for at most 2 hours.

13. The process of claim 1 wherein the catalyst is prepared using the following steps:
a) providing a cobalt amine precursor solution;
b) contacting the solution with a silica-containing support material for a period of time sufficient to allow a desired amount of cobalt to form a precipitate on the support material;
c) removing the precipitate from the solution; and
d) allowing the precipitate to dry to form a dried silica-based cobalt-deposited material, and
e) calcining the dried silica-based cobalt-deposited material, and
f) optionally, reducing the calcined silica-based cobalt-deposited material.

14. The process of claim 1 wherein said cobalt is derived from a cobalt amine carbonate precursor.

15. The process according to claim 1 wherein a first portion of said catalytically active first metal is first deposited by precipitation on said silica compound to produce a precipitate and a second portion of said catalytically active first metal is deposited on the said precipitate by impregnation.

16. The process according to claim 15 wherein the catalytically active first metal comprises cobalt.

17. The process according to claim 16 wherein the catalyst is made by the steps of:
a) providing a cobalt amine carbonate solution that contains the first portion of said catalytically active first metal;
b) contacting the solution with a silica-containing support material for a period of time sufficient to allow a desired amount of cobalt to form a precipitate on the support material;
c) removing the precipitate-loaded support from the solution; and
d) allowing the precipitate-loaded support to dry and, optionally, calcining the dried precipitate, to obtain a partially loaded support; and
e) impregnating the partially loaded support with a cobalt precursor in a solution containing the second portion of said catalytically active first metal to form a fully loaded support;
d) allowing the fully loaded support to dry; and
e) calcining the fully loaded support, and
f) optionally, reducing the calcined fully loaded support.

18. The process of claim 1, further comprising a second metal selected from the group of promoters consisting of Re, Ru, Pt, Ag, B, and combinations thereof

19. The process of claim 1, further comprising a second metal selected from the group of promoters consisting of Re, Ru, Pt, and combinations thereof.

20. The process of claim 19 wherein said second metal comprises Pt.

21. The process of claim 19 wherein said second metal content comprises up to 1 wt % of the total catalyst.

22. The process of claim 21 wherein said carrier material has an average pore size distribution of between 50-300 Å.

23. The process of claim 1 wherein said silica compound is selected from the group consisting of silica, silica-titania, silica-alumina, silica-zirconia, silica-vanadia, and silica-magnesia.

24. The process of claim 1 wherein the catalyst has a desired mechanical stability at said conversion-promoting conditions, and said mechanical stability is achieved by pre-treatment of the carrier material.

25. The process of claim 24 wherein the pre-treatment of the carrier material comprises at least one of: adding at least one structural promoter, calcination, and chemical treatment.

26. The process of claim 24 wherein the pre-treatment comprises adding at least one structural promoter to the carrier material.

27. The process of claim 24 wherein the pre-treatment comprises calcination of the carrier material at a temperature between 200 and 900° C.

28. The catalyst of claim 1 wherein said first metal comprises 5-20 wt % cobalt.

29. A Fischer-Tropsch catalyst comprising:
a catalytically active first metal comprising cobalt; and
a carrier material comprising silica or a silica compound;
wherein the catalyst has a cobalt surface area per gram catalyst of at least 13 m²/g.

30. The catalyst according to claim 29 wherein the catalyst has an alpha of at least 0.87.

31. The catalyst according to claim 29 wherein the catalyst has a CO conversion of at least 24%.

32. The catalyst according to claim 29 wherein the catalyst is prepared by an impregnation technique.

33. The catalyst according to claim 29 wherein the catalyst is prepared by a precipitation technique.

34. The catalyst according to claim 29 wherein the catalyst is prepared by a combination of a precipitation technique and an impregnation technique.

35. The catalyst of claim 29 wherein the catalyst has a desired mechanical stability at said conversion-promoting conditions, and that said mechanical stability of the catalyst is achieved by pre-treatment of the carrier material.

36. The catalyst of claim 35 wherein the pre-treatment of the carrier material comprises at least one of: addition of at least one structural promoter, calcination, and chemical treatment.

37. The catalyst of claim 35 wherein the pre-treatment comprises adding at least one structural promoter to the carrier material.

38. The catalyst of claim 35 wherein the pre-treatment comprises calcination of the carrier material at a temperature between 200 and 900° C.

39. The catalyst of claim 29 wherein said silica compound comprises silica, silica-titania, silica-alumina, silica-zirconia, silica-vanadia, and silica-magnesia.

40. The catalyst of claim 29 wherein said cobalt is derived from a cobalt amine precursor.

41. The catalyst of claim 40 wherein said cobalt amine precursor is subjected to a precipitation technique.

42. The catalyst of claim 29, further comprising a second metal selected from the group of promoters consisting of Re, Ru, Pt, Ag, B, and combinations thereof.

43. The catalyst of claim 29, further comprising a second metal selected from the group of promoters consisting of Re, Ru, Pt, and combinations thereof.

44. The catalyst of claim 43 wherein said second metal comprises Pt.

45. The catalyst of claim 43 wherein said second metal comprises up to 1 wt % of the total catalyst weight.

46. The catalyst of claim 29 wherein said carrier material comprises silica.

47. The catalyst of claim 46 wherein said carrier material has an average pore size distribution of between 50-300 Å.

48. The catalyst of claim 29 wherein said first metal content comprises 5-20 wt % cobalt.

49. The catalyst of claim 29 wherein said cobalt has a surface area of at least 16 m² per gram catalyst.

50. The catalyst of claim 49 wherein the catalyst is substantially free of cobalt silicate.

51. A process for producing a Fischer-Tropsch catalyst comprising:
a) heating a mixture comprising a silica-containing component and a cobalt amine carbonate under conditions sufficient to precipitate cobalt hydroxycarbonate on the silica-containing component to form a cobalt-loaded support;
drying the cobalt-loaded support; and
calcining the dried cobalt-loaded support.

52. The process of claim 51 wherein said cobalt amine carbonate comprises an aqueous solution.
